(19) United States
(12) Patent Application Publication
(10) Pub. No.: US 2025/0134611 A1
(43) Pub. Date: May 1, 2025

(54) MEDICAL JOYSTICK WITH RELEASE SENSOR, MEDICAL SYSTEM AND COMPUTER-IMPLEMENTED CONTROL METHOD

(71) Applicant: B. Braun New Ventures GmbH, Freiburg im Breisgau (DE)

(72) Inventors: Tim Beyl, Freiburg im Breisgau (DE); Adrian Ramos, Berlin (DE)

(21) Appl. No.: 18/922,551

(22) Filed: Oct. 22, 2024

(30) Foreign Application Priority Data
Oct. 25, 2023 (DE) 10 2023 129 470.9

(51) Int. Cl. A61B 34/00; A61B 90/90; G01D 5/24; G01S 13/42

(52) U.S. Cl. CPC A61B 34/74; A61B 90/90; G01D 5/24; G01S 13/42; A61B 2034/742

(57) ABSTRACT

A medical joystick for actuating a medical device has a base and an actuating lever movably connected to the base. The actuating lever is pivotable for an actuating movement about at least one axis, and preferably also rotatable about at least one axis. Deflection of the actuating lever about the at least one axis is identified as an actuating movement by an actuating sensor, for example as an actuator, as an actuating input. The joystick also includes at least one release sensor arranged on the base and/or the actuating lever and that identifies the presence of a user. A control unit is adapted to release the actuating input for actuation and to actuate the medical device only when the presence of the user is identified. The joystick can be used with or operated by a medical system, a control method, a computer-readable storage medium, and a computer program.

### CROSS-REFERENCE TO RELATED APPLICATION

This application claims priority under 35 U.S.C. § 119 to German Application No. 10 2023 129 470.9, filed on Oct. 25, 2023, the content of which is incorporated by reference herein in its entirety.

### FIELD

The present disclosure relates to a medical joystick (as an input means, a 3D mouse can also be regarded as a joystick) for actuating (or controlling) a medical device with a (joystick) base and a (joystick) actuating lever/operating lever/joystick handle or manipulating section or gripping section movably connected to the base as a movable lever, in particular a pivotable lever, which can be moved for an actuating movement in or about at least one axis, in particular in or about several axes (in particular exactly three axes) and preferably furthermore rotatable about at least one axis, in particular preferably several axes (in particular exactly three axes), wherein the deflection thereof in or about (pivoting) the at least one axis is identified as an actuating movement by means of an actuating sensor or deflection sensor as an actuating input. In addition, the disclosure relates to a medical system, a computer-implemented control method, a computer-readable storage medium and a computer program.

### BACKGROUND

In the field of medicine, medical devices are increasingly being controlled digitally, which requires secure and effective input. In particular, a robot with a robotic arm for gripping can be used, which is moved by a user input and whose manipulation by means of an end effector is also carried out by the user input.

One problem with the current state of the art is that unintentional operation of the joystick or movement of the robot can occur, which must be avoided at all costs and prevented, especially in a medical environment with correspondingly high risks for a patient. Unintentional control can be caused in particular by incorrect measurement data generated by the input device (joystick) itself (internal joystick error). Actuation of the joystick with corresponding deflection can also result in unintentional control due to contact with physical objects that collide with the joystick. In a particularly unfavorable case, for example, an instrument can be guided intraoperatively by a robot during an intervention and a healthcare professional can unintentionally bump into the joystick, for example when changing position, and move the instrument intraoperatively, resulting in tissue damage.

Initial concepts currently exist that make it possible to activate a joystick using a safety mechanism. On the one hand, the joystick can be activated by attaching a physical button or switch to the handle of the joystick itself, which must be pressed separately by the user gripping the joystick and held down while controlling it.

This variant of the safety mechanism has the disadvantage that a physical button must be integrated into the moving part of the joystick and wired accordingly. This safety mechanism cannot be easily integrated into existing joystick solutions or products, and in the case of highly sensitive joystick designs, such as a 3D space mouse/3D mouse (with both deflections and rotations in and around three axes), the required cables impair the sensor readings of the actuation sensor.

Another variant of a safety mechanism is a concept for safety-critical applications in which the sensor readings are measured redundantly to enable detection of a joystick failure or error. However, this concept cannot prevent accidental movements of the joystick, especially if the joystick is mounted on a robot and the joystick hits an obstacle.

U.S. Pat. No. 7,757,579 B2, for example, discloses a joystick with a deflectable handle and sensors that identify a movement of the handle when it is deflected and swiveled. A microprocessor in a base verifies the input signals before they are forwarded to a remote control. Buttons are arranged at the top of the joystick, which are guided to the base of the joystick via a channel running through the middle of the handle. There, the cable is connected to a controller as a control unit.

### SUMMARY

It is therefore the task of the present disclosure to avoid or at least reduce the disadvantages of the state of the art and, in particular, to provide a medical joystick and a medical system, a control method, a computer-readable storage medium, and a computer program with an efficient safety mechanism which ensures safe operation and, in particular, ensures that control only takes place in the event of deliberate, intentional input by a user without a joystick error.

The problems of the present disclosure are solved with respect to a medical joystick, a medical system, a control method, a computer-readable storage medium, and a computer program according to the disclosure.

Thus, a basic idea of the present disclosure is to provide a medical joystick comprising a release sensor that does not need to be explicitly actuated (and moved and pressed). The release sensor may in particular be integrated on the (joystick) handle and/or the base of the joystick to enable integration into many different joystick designs, and the release sensor may in particular utilize multiple identification modalities (or technologies), such as integrated into a single joystick itself to identify redundant releases, or different embodiments of the joysticks may have different identification modalities.

In other words, it is proposed to provide a joystick with a safety mechanism with a secondary release channel of a release sensor, which makes it possible to detect whether the joystick is being operated/used by a user or whether it has only been operated accidentally/unintentionally or even if there is an internal joystick error. Unintentional actuation can be caused by incorrect measurement data, for example incorrect data generated by the joystick itself (error), or by actuation due to contact with physical objects that collide with the joystick. The joystick described here enables safe operation of a (joystick-operated/joystick-controlled) medical device in areas where increased safety is required or where users must ensure that there is no incorrect joystick input information or joystick input data.

The joystick design described here can also be easily integrated into conventional joysticks or existing joysticks can be easily modified to integrate the security technology using the release sensor. In particular, there are two independent measurement channels that can be combined to detect incorrect values or inadvertently generated joystick extensions. In particular, the design can be made completely fail-safe to enable integration into safety-critical applications.

In still quite different words, a medical joystick for actuating a medical device is proposed with a (joystick) base and an actuating lever which is movably connected to the base and which is movable or deflectable for an actuating movement in (direction) or about at least one axis, in particular in or about several axes, in particular pivotable, and preferably furthermore rotatable about at least one axis, particularly preferably several axes, wherein its deflection in or about the at least one axis is identified as an actuating movement by means of an actuating sensor, for example as a position sensor (actuator), as an actuating input. The joystick further comprises: at least one release sensor (in particular several release sensors), which is arranged on the base and/or the actuating lever and is adapted to identify the presence of a user, in particular a hand gripping the actuating lever, and a control unit, which is adapted to release the actuating input for actuation only when the presence of the user is identified and to forward it to the medical device in order to control the medical device.

The at least one release sensor can therefore be integrated into: the base of the joystick (as the static part) and/or the movable part of the joystick, namely the actuating lever (with the handle or manipulating section). If the release sensor is provided on or in the base of the joystick, the moving part of the joystick does not need to be changed and no additional torque or force is exerted on the (return) springs or elastic elements between the base and the moving part. This helps with very sensitive joystick applications, for example, where high precision is required. However, if the release sensor is integrated into the moving part (actuating lever), it must be wired in such a way that the cables or wires of the release sensor do not exert any force on any (return) springs, similar to the wiring of buttons on the joystick.

Of course, this function of release-based actuation also works for digital medical joysticks that provide a binary actuation signal (only 1 or 0) as actuation input (i.e., without discrete or continuous deflection amplitudes as usual).

According to an aspect of the present disclosure, instead of a joystick with a deflectable actuating lever, a force-torque sensor input means or a button/switch (with a movable button) for actuating a medical device having a base and a force-torque sensor or a movable button connected to the base may be provided, which identifies this as an actuation input by means of an actuation sensor, with at least one release sensor arranged on the base and/or the force-torque sensor or the button and adapted to identify a presence of a user, and a control unit which is adapted to release the actuation input for actuation and forward it to the medical device only when the presence of the user is identified.

It should be noted that any number of release sensors can be used, such as two identical identification modalities of the release sensors at different locations (positions) or two different identification modalities at the same or different locations.

Advantageous embodiments are explained below.

According to one embodiment, the medical joystick can be adapted to be used in a surgical environment such as an operating room during a surgical intervention, in particular be designed to be sterile or sterilizable and/or meet a medical approval requirement. Instead of using a conventional joystick from the gaming sector, for example, a medical joystick is provided that meets the requirements for a surgical intervention. In particular, the materials of the outer surface of the base and the actuating lever are selected so that they can be sterilized and/or preferably the base and actuating lever form a closed sterile barrier to the outside.

Preferably, the joystick can be adapted to be encased by a sterile cover or sterile sheath, in particular it can have specially configured adapters or (coupling) interfaces to which a sterile cover can be coupled and uncoupled without tools. In particular, the joystick (or a joystick system) may have a sterile cover which is adapted to be slipped over the joystick and to encase it. In particular, the sterile cover can have a transparent section or be transparent so that a surgeon can see the encased joystick.

According to a further embodiment, the actuation sensor can be connected to a first (actuation) microcontroller via a first data connection, the release sensor can be connected to a second (release) microcontroller via a second (separate) data connection, which is different from the first data connection, and the (central) control unit of the joystick is connected both to the first microcontroller and to the second microcontroller and is adapted to forward the actuation input for actuation when the presence of the user is identified and to detect an incorrect sensor signal and output a warning message via a display or to calibrate the incorrect sensor signal when the actuation input of the actuation sensor is received but without the presence of a user. Alternatively, the joystick can preferably also have two connections instead of the central control unit, each of which allows an external connection to the first and second microcontroller, so that the medical device, for example, which has the correspondingly adapted control unit, can carry out the check and release.

In particular, the release data of the release sensor can be either:
used in a gate (barrier) implemented in a microprocessor that is part of the joystick assembly. In this case, joystick data is only forwarded to the controlled device if the release sensor is active,
or the joystick data can be forwarded to a processor for the joystick information independently of the release information. This allows the joystick to be used in a redundant hardware configuration where the joystick data is processed on a different control path to the other information. For example, the release information could be used to release the movement of the robot, in particular by a safety controller/processor, and the joystick data is used in a movement control processor that generates the target for the robot.

Preferably, the release sensor can have a camera as an optical sensor or be designed in the form of a camera (for example with an optical lens system and a downstream CMOS sensor), which identifies the presence of the user, in particular a hand of the user, in particular when the actuating lever is manipulated and arranged on it in such a way that the camera is covered by the hand, and preferably the control unit is adapted to also identify a gesture movement of the user via the camera and to send a control instruction associated with the identified gesture to the medical device by means of a comparison with a stored assignment database or a trained AI system (which is trained with corresponding gestures and associated control instructions).

The release sensor can preferably be an optical sensor for measuring the difference in light intensity when the sensor is blocked by a user gripping the joystick, or optical sensors that perform image processing of what is seen from the perspective of the optical sensor and thus the joystick, in particular identifying the user or determining a proximity or distance of the user, or optical sensors that measure the distance of the gripping user, in particular the hand, preferably by means of a TOF sensor (time-of-flight sensor).

In one embodiment, a release sensor can provide a digital signal (touched or not, binary 0 or 1), while other embodiments provide release sensors that enable the measurement of a distance or even a spatial three-dimensional resolution, in particular by means of a TOF sensor array (time-of-flight sensor array). In the case of release sensors with spatial resolution, the release sensors in the joystick can also be used as contactless gesture sensors. In addition, a release sensor that requires touch but provides spatial resolution can serve as a touch-based gesture sensor or as an input device on the surface of the joystick itself.

In particular, the release sensor can have a capacitive sensor and/or an inductive sensor or be designed in the form of a capacitive or an inductive sensor that is adapted to: detect a direct/immediate touch of a user's hand, or detect with increased sensitivity a hand at a predetermined (maximum) distance with, for example, an air gap between the sensor and the hand. Sensor technologies that include capacitive or inductive sensors that identify (direct) contact or proximity (of a hand) of a user to the sensor can be used for the release sensor, for example.

According to one embodiment, the release sensor can be designed in the form of an optical sensor, in particular in the form of a camera or a brightness sensor, or in the form of an ultrasonic sensor or a radar sensor or a laser distance sensor, in order to identify a distance between a user's hand and the actuating lever.

In particular, a single-pixel sensor or a TOF sensor array or a brightness sensor or an ultrasonic sensor or a radar sensor or a laser distance sensor can be provided as a release sensor. These different identification modalities of the release sensors make it possible in particular to determine a distance to a user's hand.

According to a further embodiment, the joystick can be adapted to identify a user ID, in particular to have an RFID transponder (reader or read/write device) that reads an RFID tag of a user when a presence is identified, in particular when the actuating lever is gripped, and the control unit can be adapted to perform a comparison/matching of the user ID with a user database stored in a memory unit (with at least the columns user ID and associated authorization, in particular also a user-specific configuration of the joystick, such as a speed specification of actuating the medical device) and to send an actuating signal to the medical device if the control entry is positive for the user ID and otherwise (if the control entry is missing or negative) to block the actuation of the medical device. The release sensor can therefore comprise a sensor for identifying a user (in particular RFID sensors) in order to restrict access to the joystick to a specific user or group of users. In particular, the actuation of the medical device can be individualized to suit the user (for example: Surgeon with user ID 1 would like the robot to move more slowly than surgeon with user ID 2 when controlled by the joystick).

Preferably, the release sensor can have a Hall sensor or a reed sensor and the medical joystick can detect a magnetic field in particular via the release sensor and preferably detect a user glove provided with a magnet for a release. In other words, the release sensor may include a Hall or reed sensor, where the joystick must be used with special gloves, for example, that include magnets for identification by the release sensor.

In particular, capacitive sensors as release sensors or inductive sensors or Hall or reed sensors and co-acting sensors that do not require line of sight can be easily and simply integrated into a joystick housing (either in the housing of the moving part or in the housing of the base (of the pedestal). Alternatively or additionally, they can also be mounted on the inside of the outer housing so that they are not visible from the outside, which makes it possible to maintain the visual appearance of an existing joystick, while optical sensors require a line of sight, for example through an appropriately provided opening or window. If the release sensor is integrated into the base of the joystick, this can be done in particular at an area of the base from which the operator's hand is visible, or a window or opening (possibly with a cover) can be integrated into the moving part through which the release sensor looks in the form of an optical sensor.

In another embodiment, the sensor may not be mounted directly on the actuating lever or on an upper section of the base, but in a lower section of the base that is rigidly connected (such as bolted) to the assembly that holds the joystick. Furthermore, the release sensor can preferably be integrated into the holder of the joystick and the base (of the pedestal).

Preferably, the medical joystick can have a minimum number of degrees of freedom of one and/or a maximum of six. In particular, a slider with positive and negative deflection (or −x and +x) is realized with only one degree of freedom of a swivel of the joystick. With six degrees of freedom, three deflections in (direction-translatory) or around three axes and three rotations around three axes are possible. In particular, a 3D mouse (3D space mouse) has six degrees of freedom for control as an actuating movement.

Further preferably, the release sensor can have or be designed as a TOF sensor array (time-of-flight field), which is adapted to detect both a 3D detection and a distance to the user, in particular his hand, in order to provide gesture control in particular in addition to identifying a distance of a user's hand and thus a presence.

According to one embodiment, the release sensor can take the form of a capacitive sensor that is connected to a metal ring that runs radially around the circumference of the actuating lever in order to detect a user's hand in its entirety/from any direction/360°. The metal ring can be integrated particularly well and form a continuous surface and thus a sterile barrier with the housing on the outside.

In particular, the medical joystick can have two or three or four cameras as release sensors, the viewing axes of which each point radially outwards on the actuating lever or are arranged radially outwards, in particular are arranged in a circumferentially equally distributed manner. The viewing axes therefore run transversely, in particular perpendicularly, to a longitudinal axis of the actuating lever in a radially outward direction and cover the direct surroundings of the actuating lever.

In particular, a release sensor can have two sensors with different identification modalities in order to ensure redundant and particularly reliable identification.

With regard to a medical (control) system, the tasks are solved in that it comprises: a medical joystick according to the present disclosure, and a medical device, in particular a medical collaborative robot or a surgical microscope, which is connected to the medical joystick and which is actuated by the medical joystick.

In particular, the medical system can have a medical glove in which a magnet is integrated so that the release sensor, which has the Hall sensor or the reed sensor, can determine the presence of the glove and thus of the user. Furthermore, an RFID tag can preferably be integrated into the glove so that each user (surgeon) can be detected and individualized control can be implemented.

In particular, the medical system may have a (separate) sterile cover specifically adapted to encase the joystick to create a sterile barrier between the non-sterile joystick and the sterile (external) environment. In this way, a healthcare professional can prepare the joystick in advance of the intervention and “pull over” and secure the sterile cover over the joystick, which the surgeon can then grasp and operate manually. In particular, the joystick has at least one (special) adapter or coupling interface and the sterile cover has at least one counter-adapter which is adapted to (respectively) interact with the adapter and to provide a tool-free coupling of the sterile cover as well as decoupling in order to form and provide a safe, repeatable as well as efficient coupling structure. Preferably, the sterile cover can have a sterile cover cloth as a basic element, in particular be designed as such, which then wraps around the joystick.

With regard to a computer-implemented control method for controlling a medical device with a medical joystick, in particular according to the present disclosure, the tasks are solved in that it performs the steps:
identification of an actuation input of a medical joystick by means of an actuation sensor, which detects an actuating movement of an actuation lever connected to a base, which is movable, in particular pivotable, for an actuating movement in or about at least one axis, in particular about several axes, and preferably furthermore rotatable about at least one axis, particularly preferably several axes, wherein its deflection in or about the at least one axis is identified as an actuating movement by means of an actuation sensor;
identification of the presence of a user by means of a release sensor located on the base and/or on the actuating lever; and
releasing, by means of a control unit, when the user's presence is identified, the actuation input and corresponding actuation of the medical device.

With regard to a computer-readable storage medium and a computer program, the tasks are solved in that the latter comprises (in each case) instructions which, when executed by a computer, cause the computer to carry out the method steps of the control method according to the present disclosure.

The disclosure relating to the medical joystick according to the present disclosure also applies to the medical system and the control method analogously, just as, conversely, the disclosure of the medical system and the control method of the present disclosure also apply to the joystick.

### BRIEF DESCRIPTION OF THE DRAWINGS

The present disclosure is explained in more detail below with reference to preferred embodiments with the aid of figures.

FIG. 1 shows a schematic front view of a medical joystick according to a preferred embodiment of a medical system of a preferred embodiment, in which a capacitive (release) sensor is arranged on the movable joystick handle;

FIG. 2 shows a schematic front view of a medical joystick according to a further embodiment, in which two optical sensors are provided as release sensors on the outside, which have an identification field of view towards the side of the actuating lever;

FIG. 3 shows a schematic sectional view of a medical joystick according to a further embodiment with a release sensor provided at the base;

FIG. 4 shows a schematic sectional view of a medical joystick according to a further embodiment with optical release sensors provided at the base;

FIG. 5 shows a schematic sectional view of a medical joystick according to a further embodiment in the form of a 3D space mouse, in which the capacitive (release) sensor is oriented upwards instead of to one side;

FIG. 6 shows a schematic sectional view of a medical joystick according to a further embodiment in the form of a 3D space mouse, in which an optical sensor is aligned upwards;

FIG. 7 shows a schematic sectional view of a medical joystick according to a further embodiment, in which the capacitive (release) sensor is oriented upwards and is provided on a base of the 3D space mouse as a joystick;

FIG. 8 shows a schematic sectional view of a medical joystick according to a further embodiment, in which an optical (release) sensor is aligned upwards and provided on a base of the 3D space mouse;

FIG. 9 shows a schematic sectional view of a medical joystick according to a further embodiment, in which optical sensors are integrated in a lower section of the base or in an assembly, which are oriented upwards;

FIG. 10 shows a schematic sectional view of a medical joystick according to a further embodiment, in which a capacitive sensor is provided on a lower section of the base or on an assembly;

FIG. 11 shows a schematic sectional view of a joystick in which the entire upper part of the handle section is movable and is hinged to the base at the bottom;

FIG. 12 shows a schematic sectional view of a joystick in which the joint is located in an upper section of the joystick; and

FIG. 13 shows a flowchart of a medical computer-implemented control method according to a preferred embodiment.

The figures are schematic in nature and are only intended to aid understanding of the present disclosure. Identical elements are marked with the same reference signs. The features of the various exemplary embodiments can be used interchangeably.

### DETAILED DESCRIPTION

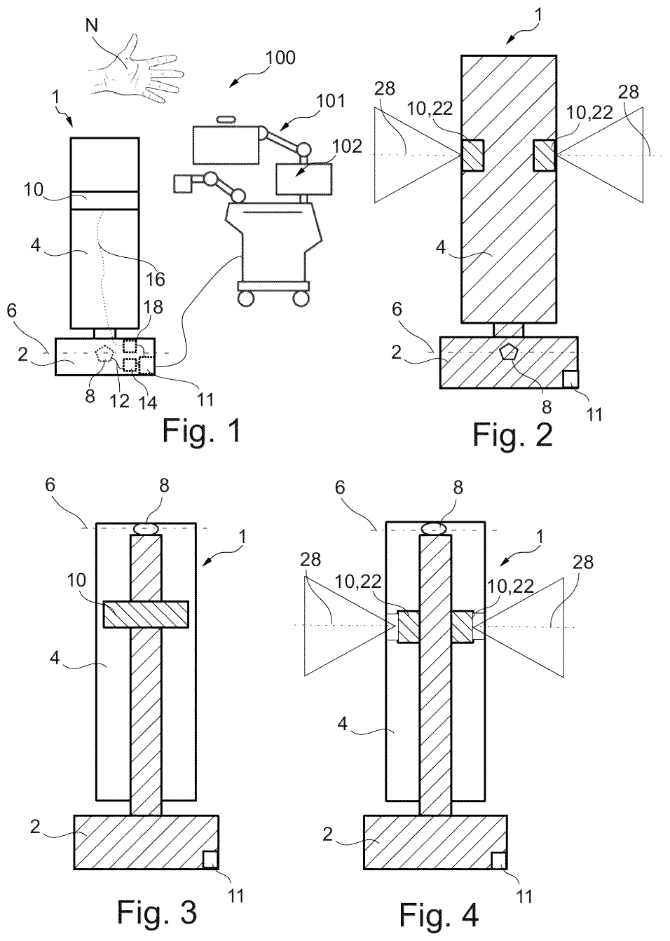

FIG. 1 shows a schematic (front) view of a medical system 100 according to a preferred embodiment with a medical joystick 1 according to a preferred embodiment of the present disclosure, and a medical device 101 in the form of a robot-guided surgical microscope connected to the medical joystick 1 by means of a cable (alternatively also possible wirelessly, for example by means of WLAN or Bluetooth), which is actuated by the medical joystick 1.

The medical joystick 1 is used for actuating the surgical microscope (as a medical device 101) and has a (joystick) base 2 (as a static component) and an actuating lever 4 or joystick handle, which is movably connected to the base 2 and articulated here, and which can be pivoted about at least one axis 6 for an actuating movement. The deflection of the actuating lever 4 (starting from a zero axis) about the at least one axis 6 as an actuating movement is identified as an actuating input by means of an actuating sensor 8. In this case, the joystick 1 can even be swiveled or rotated around three axes X, Y, Z of a Cartesian coordinate system (forward-backward, left-right, rotation around the longitudinal axis).

In contrast to the state of the art, the medical joystick 1 has at least one specially provided release sensor 10, which in the present case is arranged on the actuating lever 4 and is adapted to identify the presence of a user N. Specifically, the release sensor is adapted to identify a hand of the user N on the actuating lever 4.

For this purpose, the release sensor 10 of this embodiment has a capacitive sensor that is adapted to detect direct contact with the hand of the user N. Specifically, the capacitive sensor is connected to a metal ring 20, which extends radially around the circumference of the actuating lever 4 in order to detect a user's hand N over the entire circumference. When the metal ring 10 comes into direct contact with the user's hand, a capacitance changes and the release sensor thus detects the presence of a user N. In this embodiment, a capacitive sensor is therefore provided on the moving part of the joystick. This can be inside (on the actuating lever housing), directly outside or integrated into an actuating housing. The release sensor 10 is provided on the inside and connected to the metal ring 20 on the outside.

A control unit 11, which is arranged in the base 2 of the joysticks in the present case, is adapted to release the actuation input for actuating and to actuate the medical device 101 only when the presence of the user N is identified. Otherwise, without the “release signal” from the release sensor 10, the control unit 11 determines that the medical joystick is not being used correctly, i.e., that the joystick is being actuated without a hand being in contact with the joystick, for example by unintentionally bumping into the joystick, or that there is a potentially incorrect sensor signal from the actuation sensor, and prevents the surgical microscope from being actuated.

This makes it even safer to control a surgical microscope in an operating room during a surgical intervention, especially if a robot-guided surgical instrument is used intraoperatively instead of a surgical microscope and a movement of the robot could have fatal consequences for a patient.

In the present embodiment, the actuation sensor 8 is connected via a first data connection 12 to a first (actuation) microcontroller 14 via a cable which, for the sake of clarity, is shown as a solid line and not dashed. It is therefore specifically a separate first microcontroller 14, which is only intended for data processing of the actuation sensor and is connected to it via the cable as the first data connection 12.

Furthermore, the release sensor 10 is connected to a second (release) microcontroller 18 via a second, separate data connection 16 in the form of a cable. This means that the second microcontroller 18 is also only responsible for processing the data received from the release sensor 10. Such a configuration with separate data lines and processing is important in the field of medical technology for medical approval, as it can be used in particular to determine whether incorrect sensor values are present if, for example, the actuation sensor provides input signals without there being a deflection as an actuating movement.

The (central) control unit 11 is connected to both the first microcontroller 14 and the second microcontroller 18 (shown here as a solid line for a better view and adapted for this purpose, forwarding the actuation input to the medical device for actuation when the presence of the user N is identified by the release sensor 18 and, without the presence of a user N, detecting an incorrect sensor signal when actuation inputs are received from the actuation sensor and outputting a warning message via a display 102 of the medical device 101 or via a joystick display or warning light. Alternatively or additionally, the control unit 11 can also be adapted to calibrate the incorrect sensor signal. For example, an offset in the X direction (actuating lever 4 to the front-back) can be zeroed if there is no release for a longer period of time. This can be particularly advantageous for high-precision joysticks 1. Noise from a signal from the actuation sensor can also be identified and smoothed using a method provided in the control unit 11.

FIG. 2 shows a longitudinal sectional view of a further embodiment of a medical joystick 1 according to the present disclosure. In contrast to the joystick in FIG. 1, the joystick 1 has four cameras 22 as release sensors 10, the viewing axis 28 of which is arranged on the actuating lever 4 in a radially outward direction and is circumferentially evenly distributed. In other words, the field of vision is directed radially outwards and the viewing axes 28 are all perpendicular to a longitudinal axis of the actuating lever 4. Due to their equal distribution, the viewing axes 28 each have an angle of 90° to the neighboring viewing axis (star-shaped, in this case cross-shaped). This allows the cameras 22 to identify the presence of a user from all sides. If a user N places his hand on or around the actuating lever 4 (joystick handle), his hand covers at least one camera 22 as release sensor 10 and the presence of a user N is determined. The triangles represent the viewing angle or field of vision of the release sensors 10. The optical sensor can be a single-pixel sensor or a pixel array (as in the case of the camera 22). Both the single-pixel sensor and the pixel array can provide a spatial resolution. Thus, in another embodiment, which is not shown, a single-pixel sensor can be provided instead of the camera 22.

The cameras 22 are thus arranged in such a way that the camera 22 is covered by the user's hand when the actuating lever 4 is manipulated. In addition, the control unit 11 is adapted to identify a movement of the hand as a gesture control or gesture movement of the user when a hand of the user is identified at a minimum distance, for example greater than 10 cm, and to send a control instruction associated with the identified gesture to the medical device 101 by means of a comparison with a stored assignment database or a trained AI system. For example, the joystick 1 can be used to control a movement of the surgical microscope by moving the end effector around the axes according to the deflection, as well as when the hand is far away beyond a minimum distance and a button for gesture detection is pressed for increased safety, for example, a movement of the hand controls a further function, such as a magnification/zoom of the surgical microscope, in which a horizontal swiping movement of the hand to one side causes (actuates) a magnification and an opposite swiping movement of the hand to another side causes (actuates) a reduction of the view, which is output via the display 102.

FIG. 3 shows a further embodiment of a joystick 1 according to the present disclosure in a longitudinal sectional view, in which, in contrast to the embodiment in FIG. 1, the release sensor 1 is provided on the base 2 and does not move with the actuating lever 4. The capacitive sensor has such an increased sensitivity that it can detect a hand of the user N at a predetermined maximum distance, with an air gap and the housing of the actuating lever 4 between the release sensor 10 and the hand. This means that the release sensor can be protected from the outside environment and integrated “inside” particularly easily and securely. For example, the actuating lever 4 and the base 2 can provide a sterile sheath and can be sterilized so that they can be gripped directly by a surgeon.

FIG. 4 shows a joystick according to a further embodiment in which, in contrast to the embodiment in FIG. 1, the cameras 22 (as a pixel array) are attached to the static base 2 and look radially outwards through an opening or a viewing window. Here, too, a high-precision joystick can be provided in this way, as no cabling is required from the movable joystick handle 4 to the joystick base 2, as this runs in the base 2 itself. In this way, the camera 22 can also be designed to optimize the installation space and the weight of the camera 22 does not change the behavior of the movable joystick handle 4.

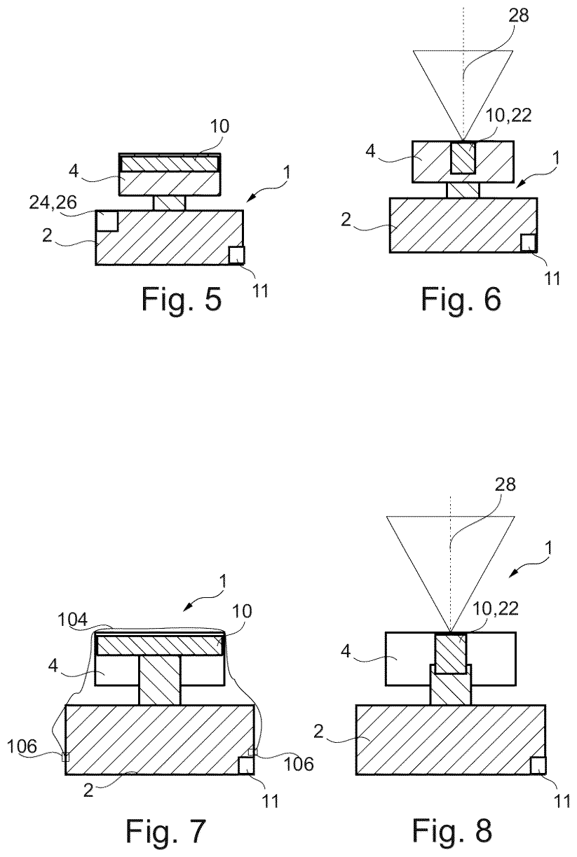

FIG. 5 shows a longitudinal sectional view of a further embodiment of a joystick 1 of the present disclosure in the form of a 3D mouse with six degrees of freedom (three translational three rotational degrees of freedom). In particular, the function and design of a 3D space mouse/3D mouse similar to the 3Dconnexion model can be provided, which is supplemented by a release sensor 10 in the form of a capacitive sensor. In the present case, the capacitive sensor is arranged on the moving part (the joystick handle) on an upper side of the 3D mouse as seen in FIG. 5 and moves with it. If a hand of the user N approaches, the capacitive sensor as release sensor 10 detects the presence and forwards the signal to the control unit 11 (in particular via a separate second data line 16 and a second microcontroller 18).

In addition, the joystick has an RFID transponder 24 and a memory unit 26 in its pedestal or lower section of the base 2, which is adapted to read an RFID tag located on a user's hand. The RFID transponder 24 reads out the RFID tag of a user N when the actuating lever is gripped or when the presence of a user's hand is identified via the release sensor 10, and the control unit 11 is adapted to perform a comparison of the (read-out) user ID with a user database stored in the memory unit 26 and to send an actuation signal to the medical device 101 if the control entry is positive for the user ID and otherwise to block the actuation of the medical device (101). This enables a personalized control release. Not only must the user N operate the joystick 1 correctly and the release sensor 10 must forward the signal, but the authorized user N must also operate the joystick to control the medical device 101. This also ensures that only people with valid certification can control the medical device.

In contrast to FIG. 5, FIG. 6 shows a further embodiment of a joystick 1 in which, instead of a capacitive sensor, a pixel array sensor is provided as a camera 22 as a release sensor 10, the orientation and viewing axis 28 of which runs coaxially to a longitudinal axis of the joysticks 1. The field of view of the camera 22 therefore covers a top area above the joystick 1. If the palm or the ball of the hand is placed on the actuating lever 4, the camera 22 identifies a presence of the hand (a dark (black) image) and determines a presence of a user N. The release sensor of FIG. 6 is similar to that of the embodiment of FIG. 4, but with the camera 22 facing upwards instead of to the side. The mounting positions of the sensors in FIG. 6 and FIG. 4 can of course also be combined, i.e., release sensor 10 as an optical sensor facing upwards and release sensor as an optical sensor facing to the side.

Instead of the camera 22, a single-pixel sensor or a TOF sensor array or a brightness sensor or an ultrasonic sensor or a radar sensor or a laser distance sensor can of course also be provided for the embodiments with a camera.

FIG. 7 shows a sectional view of a further embodiment of the joystick 1, in which, in contrast to the embodiment in FIG. 5, the capacitive sensor is attached to the base 2 and the movable part of the 3D mouse (as joystick 1) can be freely configured. In this embodiment, the joystick 1 has a separate sterile cover 104, which is thrown over the joystick 1 and is attached to the base 2 by means of an adapter 106. This means that the joystick itself can be non-sterile, but can be used in a sterile environment by means of the sterile cover 104.

FIG. 8 shows a sectional view of a further embodiment of the joystick 1 in which, in contrast to the embodiment in FIG. 6, the optical sensor is provided as a release sensor 10 on the base 2 and the movable part of the 3D mouse (as joystick 1) can be freely configured.

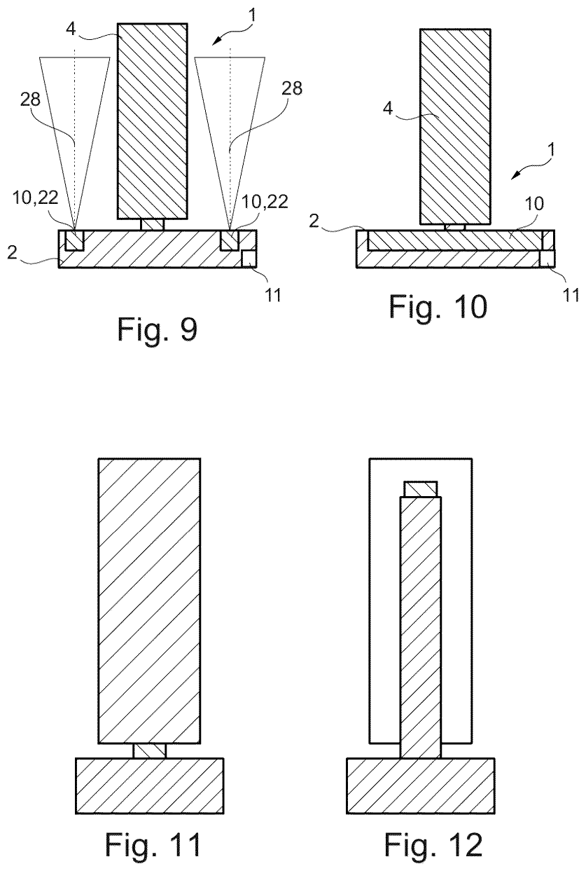

FIG. 9 shows a sectional view of a further embodiment of the present disclosure in which the release sensors 10 are integrated in the base 2 in a pedestal section of the base 2 and are oriented upwards as cameras 22 (or look from the base 2 towards the top of the joystick) and can identify a hand of a user N at a distance. Therefore, if the cameras 22 identify that the actuating lever or joystick handle 4 is being gripped by a hand, the presence of the user N is confirmed and actuation is released and executed.

FIG. 10 shows a further embodiment in which the release sensor 10 is again mounted on the lower part of the base 2 or on an assembly that holds the base 2. In this configuration, the release sensor 10 in the form of a capacitive sensor detects a hand gripping the joystick 1. The capacitive sensor is so sensitive that there can be an air gap between the release sensor 10 and the user's hand, so that the user only has to grip the actuating lever. In a further variant, however, the release sensor 10 can also be designed in such a way that direct contact by a user is required, for example for special safety-relevant interventions. Here, the user N can place one left hand directly on the release sensor 10 while gripping the actuating lever 4 with the other right hand and controlling the medical device 101, for example a robot, and moving its end effector intraoperatively.

All the joysticks in FIGS. 2 to 10 can of course be used for the medical system 100 in FIG. 1.

FIGS. 11 and 12 show two different, general designs and basic principles of a joystick. FIG. 11 shows a schematic sectional view of a joystick in which the entire upper part is movable. The entire upper movable part of the handle section is hinged to the base at the bottom. FIG. 12, on the other hand, shows a schematic sectional view of a joystick in which the joint is located in an upper section of the joystick. The base 2 extends almost to the tip and, in particular, makes it possible to mount release sensors inside the movable actuating lever 4 while they are still attached to the base 2.

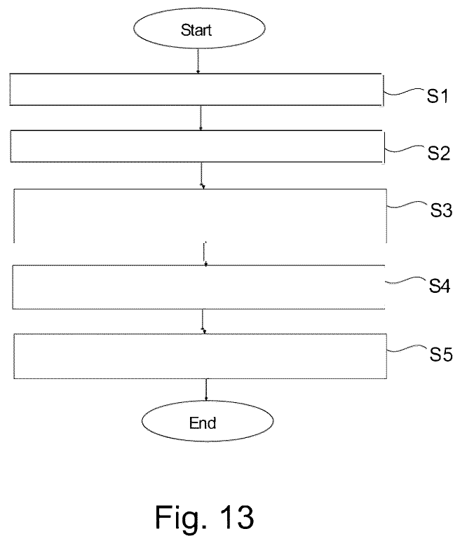

FIG. 13 shows a flowchart of a computer-implemented control method for controlling a medical device 101 with a medical joystick 1 according to the present disclosure.

In a step S1, the control method identifies an actuation input of the medical joystick 1 by means of an actuation sensor 8, which detects an actuation movement of an actuating lever 4 connected to a base 2, which is pivotable for an actuation movement about at least one axis 6, in particular about several axes, and is preferably also rotatable about at least one axis, particularly preferably several axes, its deflection about the at least one axis being identified as an actuation movement by means of an actuation sensor 8, for example as an actuator.

In step S2, the presence of a user N is identified by means of a release sensor 10, which is arranged on the base 2 and/or on the actuating lever 4.

Finally, in step S3, the control unit 11 releases the actuation input and actuates the medical device 101 when the presence of the user N is identified.

In step S4, a control instruction corresponding to the actuation movement is sent to the medical device 101 by the control unit 11 for actuation.

In an optional step S5, when the actuating lever 4 is released and a user N is not present and a period of time has elapsed, the actuation sensor is calibrated so that an actuation signal is zeroed. In this way, a possible measurement error or sensor error can be detected and rectified during operation.

List of reference symbols

| | |
|---|---|
| 1 | The medical joystick |
| 2 | (Joystick) base |
| 4 | Actuating lever (joystick handle) |
| 6 | Axis |
| 8 | Actuation sensor |
| 10 | Release sensor (user sensor) |
| 11 | Control unit |
| 12 | First data connection |
| 14 | First (actuating) microcontroller |
| 16 | Second data connection |
| 18 | Second (release) microcontroller |
| 20 | Metal ring |
| 22 | Camera |
| 24 | RFID transponder |
| 26 | Memory unit |
| 28 | Viewing axis |
| 100 | The medical system |
| 101 | The medical device |
| 102 | Display |
| 104 | Sterile cover |
| 106 | Adapter |
| N | User |
| S1 | Step Identifying an actuation input |
| S2 | Step Identifying that a user is present |
| S3 | Step Releasing the actuation of medical device |

1. A medical joystick for actuating a medical device, the medical joystick comprising:
a base;
an actuating lever;
at least one release sensor arranged on the base and/or the actuating lever and adapted to identify a presence of a user; and
a control unit which is adapted to release an actuation input for actuation and to actuate the medical device only when the presence of the user is identified,
the actuating lever movably connected to the base and movable for an actuating movement in or about at least one axis,
deflection of the actuating lever in or about the at least one axis being identified as an actuating movement by an actuating sensor as an actuating input.

2. The medical joystick according to claim 1, wherein the actuating lever is pivotable for an actuating movement in or about the at least one axis.

3. The medical joystick according to claim 1, wherein the actuating lever is movable for the actuating movement in or about a plurality of axes.

4. The medical joystick according to claim 1, wherein the medical joystick is adapted to be used in a surgical environment such as an operating room during a surgical intervention.

5. The medical joystick according to claim 4, wherein the medical joystick is designed to be sterile or sterilizable and/or fulfils a medical approval requirement.

6. The medical joystick according to claim 1, wherein:
the actuating sensor is connected to a first microcontroller via a first data connection,
the at least one release sensor is connected to a second microcontroller via a second data connection, and
the control unit is connected to the first microcontroller and to the second microcontroller,
the control unit being adapted to:
forward the actuation input for actuation when the presence of the user is identified,
detect an incorrect sensor signal, and
output a warning message via a display or a warning light or to calibrate the incorrect sensor signal when an actuation input of the actuating sensor is received but without the presence of a user.

7. The medical joystick according to claim 1, wherein the at least one release sensor has a camera as an optical sensor that identifies a presence of the user.

8. The medical joystick according to claim 7, wherein the optical sensor is configured to identify a hand of the user when the actuating lever is manipulated and arranged on the hand in such a way that the camera is covered by the hand.

9. The medical joystick according to claim 7, wherein the control unit is adapted to identify a gesture movement of the user via the camera and to send a control instruction associated with a gesture to the medical device by a comparison with a stored assignment database or a trained AI system.

10. The medical joystick according to claim 1, wherein the at least one release sensor has a capacitive sensor adapted to:
detect a direct touch of a user's hand, or
detect the user's hand at a predetermined distance with an air gap between the capacitive sensor and the user's hand with increased sensitivity.

11. The medical joystick according to claim 1, wherein the at least one release sensor is an optical sensor, a brightness sensor, an ultrasonic sensor, a radar sensor or a laser distance sensor, the at least one release sensor operable to identify a distance between a user's hand and the actuating lever.

12. The medical joystick according to claim 1, wherein the medical joystick is adapted to identify a user ID, and the control unit is adapted to perform a comparison of the user ID with a user database stored in a memory unit and to send an actuation signal to the medical device when a control entry is positive for the user ID and to block actuation of the medical device when the control entry is negative or missing for the user ID.

13. The medical joystick according to claim 1, wherein the at least one release sensor has a time-of-flight sensor array adapted to detect both a 3D detection and a distance to a user's hand.

14. The medical joystick according to claim 1, wherein the at least one release sensor is a capacitive sensor connected to a metal ring that extends radially around a circumference of the actuating lever in order to fully detect a user's hand.

15. The medical joystick according to claim 1, wherein the at least one release sensor comprises a plurality of cameras having a viewing axis arranged on the actuating lever in a radially outward direction.

16. A medical system comprising:
a medical joystick according to claim 1; and
a medical device connected to the medical joystick by data technology, the medical device being actuated by the medical joystick.

17. The medical system according to claim 16, wherein the medical system has a sterile cover adapted to encase the medical joystick.

18. A computer-implemented control method for controlling a medical device with a medical joystick according to claim 1, the computer-implemented control method comprising the steps of:
identifying an actuation input of the medical joystick by the actuating sensor that detects an actuation movement of the actuating lever;
identifying the presence of the user by the at least one release sensor; and
releasing the actuation input and actuation of the medical device by the control unit when the presence of the user is identified.

19. A computer-readable storage medium comprising instructions which, when executed by a computer, cause the computer to perform the computer-implemented control method according to claim 18.

20. A computer program comprising instructions which, when executed by a computer, cause the computer to perform the computer-implemented control method according to claim 19.
